(19) United States
(12) Patent Application Publication
(10) Pub. No.: US 2020/0138892 A1
(43) Pub. Date: May 7, 2020

(54) ANTI-DIABETIC ACTIVITY OF NEEM EXTRACT AND SYNERGISTIC COMBINATIONS OF UROLITHINS A AND B

(71) Applicant: Natreon, Inc., New Brunswick, NJ (US)

(72) Inventors: Muruganandam A. Veeraragavan, Pondicherry (IN); Chandan K. Sen, Indianapolis, IN (US); Sanyasi R. Kalidindi, Monroe, NJ (US)

(73) Assignee: Natreon, Inc., New Brunswick, NJ (US)

(21) Appl. No.: 16/672,743

(22) Filed: Nov. 4, 2019

(30) Foreign Application Priority Data
Nov. 5, 2018 (IN) 201841041788

(51) Int. Cl. A61K 36/58; A61K 9/08; A61P 3/10

(52) U.S. Cl. CPC A61K 36/58; A61P 3/10; A61K 9/08

(57) ABSTRACT

Methods of treating symptoms of type 2 diabetes in a human subject by administering a composition comprising urolithin A and urolithin B, are provided. Methods of treating symptoms of type 2 diabetes and metabolic syndrome in a human subject by administering a composition comprising neem extract are provided.

This application claims priority to Indian Provisional application 201841041788, filed on Nov. 5, 2018, which is hereby incorporated by reference herein.

### FIELD OF INVENTION

The present invention relates to improvement of symptoms of type 2 diabetes and metabolic syndrome using neem extract. This invention also relates to improvement of symptoms of type 2 diabetes using Urolithin A and Urolithin B in combination. This invention also relates to a method of reducing blood glucose levels using neem extract and using Urolithin A and Urolithin B in combination. A synergistic combination of Urolithin A (3,8-dihydroxy-dibenzo-α-pyrone) and Urolithin B (3-hydroxy-dibenzo-α-pyrone) is provided in a particularly effective ratio. A neem extract in a particular concentration is also provided.

### BACKGROUND

Diabetes is reaching epidemic proportions throughout the world. Despite the availability of several prescription medicines available for managing diabetes, there is a constant need for better medications with fewer or no side effects and additional benefits. Natural products may fill such a void, not just because they are natural products, but because humans have co-evolved with the plants surrounding them and, as a result of which, their bodies may respond better to the holistic curative properties of the natural products. Two classes of such natural products are: (1) Neem (Azadirachta indica) extracts; and (2) Urolithins A and B, also known chemically as 3,8-dihydroxy-dibenzo-α-pyrone and 3-hydroxy-dibenzo-α-pyrone, respectively.

Urolithins are bioactives present in Shilajit, which is derived from a humic exudate from sedimentary rocks. New research shows that these molecules are also metabolites of ellagitannins, generated by the microbiome in the gastrointestinal tract of the animals. In the present study, it was surprisingly found that the combination of 3,8-dihydroxy-dibenzo-α-pyrone and 3-hydroxy-dibenzo-α-pyrone, in a particular ratio, showed a synergistic significant anti-diabetic effect, while the individual urolithins have no significant effect, in genetically diabetic animals. In streptozotocin-induced diabetic animals, both urolithins showed anti-diabetic activity.

Azadirachta indica, commonly known as neem, nitree, or Indian lilac, is a tree in the mahogany family Meliaceae. It is one of two species in the genus Azadirachta, and is native to the Indian subcontinent, i.e., India, Nepal, Pakistan, Bangladesh, Sri Lanka, and Maldives. It is typically grown in tropical and semi-tropical regions. Neem trees also grow in islands located in the southern part of Iran. Its fruits and seeds are the source of neem oil.

Products made from neem trees have been used in India for over two millennia for their medicinal properties. Neem products are considered a major component in siddha medicine and Ayurvedic and Unani medicine and is particularly prescribed for skin diseases. Neem oil is also used for healthy hair, to improve liver function, detoxify the blood, and balance blood sugar levels. Neem leaves have also been used to treat skin diseases like eczema and psoriasis.

Neem extracts have been the subject of many pre-clinical studies, including studies on diabetes. In the present study, an aqueous extract of neem leaves and twigs has been evaluated for its anti-diabetic activity in streptozotocin-induced diabetic animals as well as in genetically diabetic animals. Many products work on streptozotocin-induced diabetic animals, but not on genetically insulin-deficient animals; however, the extract of the present application worked in both types of animals.

Diabetes mellitus type 2 (also known as type 2 diabetes) is a long-term metabolic disorder that is characterized by high blood sugar, insulin resistance, and relative lack of insulin. See, e.g., Causes of Diabetes, NATIONAL INSTITUTE OF DIABETES & DIGESTIVE & KIDNEY DISEASES (June 2014), incorporated by reference herein in its entirety. Common symptoms include increased thirst, frequent urination, and unexplained weight loss. See, e.g., Diagnosis of Diabetes and Prediabetes, NATIONAL INSTITUTE OF DIABETES & DIGESTIVE & KIDNEY DISEASES (June 2014), incorporated by reference herein in its entirety. Symptoms may also include increased hunger, feeling tired, and sores that do not heal. Often symptoms come on slowly. Long-term complications from high blood sugar include heart disease, strokes, diabetic retinopathy which can result in blindness, kidney failure, and poor blood flow in the limbs which may lead to amputations. See, e.g., Diabetes Fact Sheet, WORLD HEALTH ORGANIZATION (August 2011), incorporated by reference herein in its entirety. The sudden onset of hyperosmolar hyperglycemic state may occur; however, ketoacidosis is uncommon. See, e.g., F. J. Pasquel & G. E. Umpierrez, Hyperosmolar hyperglycemic state: a historic review of the clinical presentation, diagnosis, and treatment, 37 DIABETES CARE 3124 (2014); O. A. Fasanmade, et al., Diabetic ketoacidosis: diagnosis and management, 37 AFRICAN J. OF MEDICINE & MEDICAL SCIS. 99 (2008); each of which is incorporated by reference herein in its entirety.

Type 2 diabetes primarily occurs as a result of obesity and lack of exercise. Some people are more genetically at risk than others. Type 2 diabetes makes up about 90% of cases of diabetes, with the other 10% due primarily to diabetes mellitus type 1 and gestational diabetes. In diabetes mellitus type 1, there is a lower total level of insulin to control blood glucose, due to an autoimmune induced loss of insulin-producing beta cells in the pancreas. See, e.g., THE AUTOIMMUNE DISEASES 575 (Ian MacKay & Noel Rose, eds., Academic Press 2014); Chapter 17: Pancreatic hormones & diabetes mellitus, in GREENSPAN'S BASIC & CLINICAL ENDOCRINOLOGY (9th ed., David G. Gardner & Dolores Shoback, eds., McGraw-Hill Medical 2011); each of which is incorporated by reference herein in its entirety. Diagnosis of diabetes is by blood tests such as fasting plasma glucose, oral glucose tolerance test, or glycated hemoglobin (A1C).

Type 2 diabetes is partly preventable by staying a normal weight, exercising regularly, and eating properly. Treatment involves exercise and dietary changes. If blood sugar levels are not adequately lowered, the medication metformin is typically recommended. See, e.g., N. M. Maruthur, et al., Diabetes Medications as Monotherapy or Metformin-Based Combination Therapy for Type 2 Diabetes: A Systematic Review and Meta-analysis, 164 ANNALS OF INTERNAL MEDICINE 740 (2016); A. Saenz, et al., Metformin monotherapy for type 2 diabetes mellitus, COCHRANE DATABASE OF SYSTEMATIC REVIEWS (2005); each of which is incorporated by reference herein in its entirety. Many people may eventually also require insulin injections. See, e.g., A. J. Krentz & C. J. Bailey, Oral antidiabetic agents: current role in type 2 diabetes mellitus, 65 DRUGS 385 (2005), incorporated by reference herein in its entirety. In those on insulin, routinely checking blood sugar levels is advised; however, this may not be needed in those taking antidiabetic pills. See, e.g., U. L. Malanda, et al., Self-monitoring of blood glucose in patients with type 2 diabetes mellitus who are not using insulin, COCHRANE DATABASE OF SYSTEMATIC REVIEWS (2012), incorporated by reference herein in its entirety. Bariatric surgery often improves diabetes in those who are obese. See, e.g., S. Cetinkunar, et al., Effect of bariatric surgery on humoral control of metabolic derangements in obese patients with type 2 diabetes mellitus: How it works, 3 WORLD J. OF CLINICAL CASES 504 (2015); S. Ganguly, et al., Metabolic bariatric surgery and type 2 diabetes mellitus: an endocrinologist's perspective, 29 J. OF BIOMEDICAL RESEARCH 105 (2015); each of which is incorporated by reference herein in its entirety.

Rates of type 2 diabetes have increased markedly since 1960 in parallel with obesity. See, e.g., Susan Moscou, Getting the word out: advocacy, social marketing, and policy development and enforcement, in MARIE TRUGLIO-LONDRIGAN & SANDRA B. LEWENSON, PUBLIC HEALTH NURSING: PRACTICING POPULATION-BASED CARE 317 (2d ed., Jones & Bartlett Learning 2013), incorporated by reference herein in its entirety. As of 2015, there were approximately 392 million people diagnosed with the disease, compared to around 30 million in 1985. See, e.g., GBD 2015 Disease and Injury Incidence and Prevalence, Collaborators, Global, regional, and national incidence, prevalence, and years lived with disability for 310 diseases and injuries, 1990-2015: a systematic analysis for the Global Burden of Disease Study 2015, 388 THE LANCET 1545 (2016); S. Smyth & A. Heron, Diabetes and obesity: the twin epidemics, 12 NATURE MEDICINE 75 (2006); each of which is incorporated by reference herein in its entirety. Typically, it begins in middle or older age, although rates of type 2 diabetes are increasing in young people. See, e.g., H. Tfayli & S. Arslanian, Pathophysiology of type 2 diabetes in youth: the evolving chameleon, 53 ARQUIVOS BRASILEIROS DE ENDOCRINOLOGIA & METABOLOGIA 165 (2009); Giuseppina Imperatore, et al., Projections of Type 1 and Type 2 Diabetes Burden in the U.S. Population Aged<20 Years Through 2050, 35 DIABETES CARE 2515 (2012); each of which is incorporated by reference herein in its entirety. Type 2 diabetes is associated with a ten-year-shorter life expectancy. See, e.g., WILLIAMS TEXTBOOK OF ENDOCRINOLOGY 1371 (12th ed., Shlomo Melmed, et al., eds., Elsevier/Saunders), incorporated by reference herein in its entirety.

Despite the availability of several prescription medicines available for managing diabetes, there is a constant need for better medications with fewer or no side effects and additional benefits. Natural products may fill such a void, not just because they are natural products, but because humans have co-evolved with the plants surrounding them and, as a result of which, their bodies may respond better to the holistic curative properties of natural products. Two classes of such natural products are: (1) Neem (Azadirachta indica) extracts; and (2) Urolithins A and B, also known chemically as 3,8-dihydroxy-dibenzo-α-pyrone and 3-hydroxy-dibenzo-α-pyrone, respectively.

### SUMMARY

In one embodiment, a method for treating or preventing symptoms of type 2 diabetes is described, comprising administering to an individual in need thereof a therapeutically effective amount of a composition comprising an aqueous extract of Azadirachta indica, wherein post-prandial blood glucose or fasting blood glucose is reduced, and/or HbA1c is reduced, compared to placebo. In one embodiment a method for treating or preventing symptoms of metabolic syndrome is described, comprising administering to an individual in need thereof a therapeutically effective amount of a composition comprising an aqueous extract of Azadirachta indica, wherein post-prandial blood glucose or fasting blood glucose is reduced, and endothelial function is improved compared to placebo, while HbA1c and hsCRP decreased compared to baseline.

In another embodiment, a method of treating or preventing endothelial dysfunction in a diabetic individual suffering from type 2 diabetes mellitus is described, comprising administering to the individual in need thereof a therapeutically effective amount of a composition comprising an aqueous extract of Azadirachta indica, wherein endothelial function is improved.

In another embodiment, a method for treating or preventing symptoms of type 2 diabetes is described, comprising administering to an individual in need thereof a therapeutically effective amount of a composition comprising urolithin A and urolithin B in combination.

### BRIEF DESCRIPTION OF THE DRAWINGS

FIG. 1 depicts a bar graph representing the effect on fasting blood glucose (“FBG”) after 0, 4, 5, 6, and 7 weeks of supplementation with neem extract (50 mg/kg), urolithin A (10 mg/kg), and urolithin B (2 mg/kg). Bars represent groups' mean±SD (n=3-5 in each group). The bar chart legend as read from left to right, as follows, corresponds to the order of the data bars, viewed left to right: (1) placebo, (2) neem, (3) 3,8-di-OH-DBP, (4) 3-OH-DBP, (5) 3,8-di-OH-DBP+3-OH-DBP.

### DETAILED DESCRIPTION

Neem products are considered a major component in siddha medicine and Ayurvedic and Unani medicine and are particularly prescribed for skin diseases. Neem oil is also used for healthy hair, to improve liver function, detoxify the blood, and balance blood sugar levels. Neem leaves have also been used to treat skin diseases like eczema and psoriasis. Neem extracts have been the subject of many pre-clinical studies, including studies on diabetes.

In yet another aspect, the present invention demonstrates the usefulness of neem extract in treating human individuals suffering from symptoms of diabetes, including, but not limited to, high blood glucose levels.

Neem (Azadirachta indica) tree is considered divine in India and is ubiquitous in the Indian subcontinent. Different parts of the tree offer different bioactive constituents, which include azadirachtin, nimbolinin, nimbin, nimbidin, nimbidol, sodium nimbinate, gedunin, salannin, and quercetin. Leaves contain ingredients such as nimbin, nimbanene, 6-desacetylnimbinene, nimbandiol, nimbolide, ascorbic acid, n-hexacosanol and amino acid, 7-desacetyl-7-benzoylazadiradione, 7-desacetyl-7-benzoylgedunin, 17-hydroxyazadiradione, and nimbiol. Quercetin and β-sitosterol, polyphenolic flavonoids, fresh leaves are known to have antibacterial and antifungal properties and seeds hold valuable constituents including gedunin and azadirachtin (See, Mohammad A. Alzohairy, “Therapeutics Role of Azadirachta indica (Neem) and its Active Constituents in Diseases Prevention and Treatment,” Evidence-Based Complementary and Alternative Medicine, Volume 2016, Article ID 7382506, 11 pages). Different parts of the plant and different extraction solvents and methods yield extracts having different bioactive composition. Numerous biological and pharmacological activities have been reported including antibacterial, antifungal, and anti-inflammatory, hypoglycemic, anti-gastric ulcer, and anti-cancer activities. Neem modulates the activity of various tumour suppressor genes (e.g., p53, pTEN), angiogenesis (VEGF), transcription factors (e.g., NF-κB), and apoptosis (e.g., bcl2, bax). Neem also plays role as an anti-inflammatory via regulation of proinflammatory enzyme activities including cyclooxygenase (COX), and lipoxygenase (LOX) enzyme.

In an embodiment of the present invention, PhytoBGS® available from Natreon, Inc. is an aqueous extract of Azadirachta indica (Neem) leaves and twigs (in 1:1 w/w ratio) for blood glucose management. It has been shown in a 12-week long, randomized, double-blend, placebo-controlled clinical study in prediabetics to significantly decrease the fasting as well as the post-prandial blood glucose levels and reflection index, a measure of endothelial function, by the end of 12 weeks, at 500 mg BID dose, compared to the placebo group. At this dose, it also significantly decreased HOMA insulin resistance, glycosylated hemoglobin (HbA1c) levels, the oxidative stress biomarkers, hsCRP, IL-6 and TNF-α, when compared to the baseline values.

PhytoBGS® has also been shown herein in another 12-week long, randomized, double-blend, placebo-controlled clinical study in type 2 diabetics (T2DM), who have been already on a stable dose of metformin, to significantly improve fasting and post-prandial blood glucose levels, HOMA Insulin Resistance, HbA1c, hsCRP and all the oxidative stress parameters, at all the doses studied—125 mg, 250 mg and 500 mg BID, at the end of 12 weeks. In the present study, significant results were obtained at 4 weeks and 8 weeks as well with many of the parameters tested.

In one embodiment, the present invention describes a composition comprising urolithin A and urolithin B, wherein the wt./wt. ratio of urolithin B to urolithin A is from about 0.2:1 to about 1:1. Pharmaceutical or nutraceutical compositions may be prepared by including an acceptable carrier or excipient.

In an alternative embodiment, the present invention describes a composition comprising neem extract. Pharmaceutical or nutraceutical compositions may be prepared by including an acceptable carrier or excipient.

In an embodiment, the pharmaceutical compositions of the present invention are for use in a method for treating symptoms of diabetes in a human subject.

The symptoms of diabetes can include, but are not limited to, high blood glucose levels.

Shilajit is composed of rock humus, rock minerals, and organic substances that have been compressed by layers of rock mixed with marine organisms and microbial metabolites. It oozes out of the rocks in the Himalayas, at higher altitudes ranging from 1000-5000 meters, as black mass, and is regarded as a maharasa (super-vitalizer) in Ayurveda, the traditional Indian system of medicine, dating back to 3500 B. C. Shilajit contains fulvic acids as the main components, along with dibenzo-α-pyrones (“DBPs”) and dibenzo-α-pyrone chromoproteins.

Two primary DBP constituent components of Shilajit are 3,8-dihydroxy-dibenzo-α-pyrone (also known as “Urolithin A,” or, alternatively, “3,8-(OH)₂-DBP”) and 3-hydroxy-dibenzo-α-pyrone (also known as “Urolithin B,” or, alternatively, “3-(OH)-DBP”). Both of these compounds were custom-synthesized to 99% purity at contract manufacturing sites for Natreon, Inc.

Fulvic acid complex, derived from Shilajit, is an assembly of naturally occurring low and medium molecular weight compounds comprising oxygenated dibenzo-α-pyrones (DBPs), both in reduced as well as in oxidized form, as the core nucleus, and acylated DBPs and lipids as partial structural units, along with fulvic acids (“FAs”). Fulvic acid complex material derived from alluvial sources lack DBPs; instead, the core nucleus of alluvial fulvic acid is comprised of benzoic acid.

Thus, the active components of Shilajit contain dibenzo-α-pyrones and related metabolites, small peptides (constituting non-protein amino acids), some lipids, and carrier molecules (fulvic acids). See, e.g., S. Ghosal, et al., Shilajit Part 1 Chemical constituents, 65 J. PHARM. SCIs. 772 (1976); S. Ghosal, et al., Shialjit Part 7 Chemistry of Shilajit, an immunomodulatory ayurvedic rasayana, 62 PURE APPL. CHEM. (IUPAC) 1258 (1990); S. Ghosal, et al., The core structure of Shilajit humus, 23 SOIL BIOL. BIOCHEM. 673 (1992); U.S. Pat. No. 6,440,436 (and references cited therein); U.S. Pat. No. 6,869,612 (and references cited therein); each of which is incorporated by reference herein in its entirety.

Shilajit (e.g., PrimaVie®) finds extensive use in Ayurveda, for diverse clinical conditions. For centuries, people living in the isolated villages in Himalayas and adjoining regions have used Shilajit alone, or in combination with other plant remedies, to prevent and combat problems with diabetes. See, e.g., V. P. Tiwari, et al., An interpretation of Ayurvedica findings on Shilajit, 8 J. RES. INDIGENOUS MED. 57 (1973), incorporated by reference herein in its entirety. Moreover, being an antioxidant, it will prevent damage to the pancreatic islet cell induced by the cytotoxic oxygen radicals. See, e.g., S. K. Bhattacharya, Shilajit attenuates streptozotocin induced diabetes mellitus and decrease in pancreatic islet superoxide dismutase activity in rats, 9 PHYTOTHER. RES. 41 (1995); S. K. Bhattacharya, Effects of Shilajit on biogenic free radicals, 9 PHYTOTHER. RES. 56 (1995); S. Ghosal, et al., Interaction of Shilajit with biogenic free radicals, 34B INDIAN J. CHEM. 596 (1995); each of which is incorporated by reference herein in its entirety. It has been proposed that the derangement of glucose, fat, and protein metabolism during diabetes, results in the development of hyperlipidemia. In one study, Shilajit produced significant beneficial effects in lipid profile in rats. See, e.g., N. A. Trivedi, et al., Effect of Shilajit on blood glucose and lipid profile in alloxan-induced diabetic rats, 36 INDIAN J. PHARMACOL. 373 (2004), incorporated by reference herein in its entirety.

As discussed, Shilajit has been used to treat various ailments. It is also recommended as a performance enhancer. Fulvic acid (FAs) are reported to elicit many important roles in biological systems of plants, in animals, as well as in humans, including: (a) improvement of bioavailability of minerals and nutrients; (b) serve as electrolytes; (c) detoxification of toxic substances including heavy metals; (d) perform as antioxidants; and (e) improvement of immune function. Furthermore, dibenzo-α-pyrones have been hypothesized to participate in the electron transport inside the mitochondria, thus facilitating production of more ATP, leading to increased energy.

In another aspect, the present invention demonstrates the usefulness of 3-hydroxy-dibenzo-α-pyrone (3-OH-DBP), 3,8-dihydroxy-dibenzo-α-pyrone (3,8-(OH)₂-DBP), or combinations thereof, in treating human individuals suffering from symptoms of diabetes, including, but not limited to, high blood glucose levels.

The following non-limiting examples are provided to illustrate the invention and are applicable to other embodiments of the present invention. The person skilled in the art will appreciate that it may be necessary to vary the procedures for any given embodiment of the invention, e.g., vary the order or steps and/or the amounts of ingredients used.

In order to evaluate the antidiabetic effects of 3,8-dihydroxy-dibenzo-α-pyrone (Urolithin A), 3-hydroxy-dibenzo-α-pyrone (Urolithin B), Shilajit, and neem extract, the following experiments were conducted on both the neem extract and the urolithins in streptozotocin-induced diabetic animals as well as genetically diabetic animals:

Study 1: Effect of Neem Extract on Streptozotocin-Induced Diabetes in Male and Female Mice;

Study 2: Effect of 3,8-Dihydroxy-Dibenzo-α-Pyrone (Urolithin A) and 3-Hydroxy-Dibenzo-α-Pyrone (Urolithin B), and their Combination in Particular Ratios, on Streptozotocin-Induced Diabetes in Male and Female Mice;

Study 3: Effect of Neem Extract, 3,8-Dihydroxy-Dibenzo-α-Pyrone (Urolithin A), 3-Hydroxy-Dibenzo-α-Pyrone (Urolithin B), and the Combination of Urolithin a and Urolithin B in Particular Ratios, on Blood Glucose Levels in Genetically Diabetic Mice.

### Example 1 (Study 1)

Objective of Study 1: To study the effect of neem extract on streptozotocin-induced diabetes in male and female mice.

Body weight, food intake, water intake, and fasting basal blood glucose level were measured before induction on diabetes. Diabetes was induced by single administration of streptozotocin (65 mg/kg body weight) in sodium citrate buffer, pH 4.5, intraperitoneally. Five days after streptozotocin administration, fasting blood glucose level was estimated. Mice having blood glucose levels of 230±20 mg % were considered for the study. The male and female mice each were divided into the following groups:

1. Vehicle Control (“Veh Con”)

2. Streptozotocin (“STZ”)

3. Neem 50+STZ

4. Neem 100+STZ

5. Neem 250+STZ

6. Metformin 250+STZ

Following streptozotocin-induced hyperglycemia, the treatment regimen was carried out for two weeks. Neem extract was suspended in 0.3% sodium carboxymethylcellulose (“CMC”) and administered at a dose of 50 mg/kg b.w., 100 mg/kg b.w., and 250 mg/kg b.w. orally for two weeks. Similarly, metformin was suspended in 0.3% CMC and administered at a dose of 250 mg/kg b.w. orally for two weeks. During the treatment schedule, body weight and food and water intake were monitored at regular intervals. Fasting blood glucose (“FBG”), HbA1C, plasma cholesterol, and triglyceride levels were monitored every week for two weeks. The results were expressed in terms of mean±SEM (n=4, from each gender). The data were subjected to one-way ANOVA followed by Tukey's test using GraphPad Prism 4.0 software to establish statistical significance (*p<0.05, **p<0.01, ***p<0.001; ^{#} p<0.05, ^{##} p<0.01, ^{###} p<0.001).

Table 1. Effect of Neem Extract on Blood Sugar Level of Streptozotocin-Induced Male Diabetic Mice.

TABLE 1

| | Vehicle Control | STZ | STZ + Neem 50 | STZ + Neem 100 | STZ + Neem 250 | STZ + Met 250 |
|---|---|---|---|---|---|---|
| | (mg %) | (mg %) | (mg %) | (mg %) | (mg %) | (mg %) |
| Day 0 | 104.4 ± 11.14 | 133 ± 27.15 | 126.6 ± 15.52 | 127.7 ± 17.23 | 105 ± 16.1 | 117.3 ± 23.99 |
| Week 1 | 120.6 ± 19.58 | 234.9 ± 16.05*** | 248.6 ± 13.47*** | 222 ± 13.24*** | 218.6 ± 24.36 | 269.2 ± 25.37*** |
| Week 2 | 107.8 ± 14.56 | 209.6 ± 10.14*** | 170.9 ± 16.57***^{, #} | 161.1 ± 18.83**^{, #} | 160.7 ± 10.02**^{, ##} | 133.4 ± 7.96**^{, #} |
| Week 3 | 128.9 ± 2.34 | 203.6 ± 5.09*** | 172.7 ± 7.34***^{, #} | 178.5 ± 0.77***^{, #} | 148.4 ± 4.73*^{, ##} | 136.2 ± 4.89^{###} |

*p < 0.05,
**p < 0.01,
***p < 0.001 in comparison to vehicle control;
^{#}p < 0.05,
^{##}p < 0.01,
^{###}p < 0.001 in comparison to STZ induced diabetic male mice

FBG (mg %) of male Neem+STZ and Metformin+STZ treated mice were compared with vehicle control mice and streptozotocin-induced diabetic mice. FBG level was significantly (***p<0.001) increased in the STZ-induced diabetic male mice. Treatment with neem extract (Neem 50, Neem 100, Neem 250) and metformin (Met 250) significantly (^{#} p<0.05, ^{##} p<0.01, ^{###} p<0.001) attenuated the STZ-induced increased sugar level.

Table 2. Effect of neem extract on blood sugar level of streptozotocin-induced female diabetic mice.

TABLE 2

| | Vehicle Control | STZ | STZ + Neem 50 | STZ + Neem 100 | STZ + Neem 250 | STZ + Met 250 |
|---|---|---|---|---|---|---|
| | (mg %) | (mg %) | (mg %) | (mg %) | (mg %) | (mg %) |
| Day 0 | 128.6 ± 4.67 | 130.3 ± 5.28 | 132.4 ± 3.62 | 96.23 ± 10.43 | 122.6 ± 10.42 | 119.2 ± 15.5 |
| Week 1 | 139.8 ± 9.06 | 303.3 ± 23.82*** | 256.6 ± 7.81*** | 236.8 ± 14.6*** | 250.6 ± 5.54*** | 241.1 ± 22.19*** |
| Week 2 | 123.1 ± 23.35 | 206.6 ± 3.64** | 157 ± 4.86*^{, #} | 149.8 ± 5.27^{##} | 148.9 ± 12.71^{##} | 133.8 ± 9.04^{##} |
| Week 3 | 139.2 ± 6.33 | 207.6 ± 3.06** | 156.8 ± 14.95*^{, #} | 145.1 ± 13.35^{##} | 127.6 ± 13.7^{###} | 121.2 ± 8.192^{###} |

**p < 0.01,
***p < 0.001 in comparison to vehicle control;
^{#}p < 0.05,
^{##}p < 0.01,
^{###}p < 0.001 in comparison to STZ-induced diabetic female mice

FBG (mg %) of female Neem+STZ and Metformin+STZ treated mice were compared with vehicle control mice and streptozotocin-induced diabetic mice. FBG level was significantly (***p<0.001) increased in the STZ-induced diabetic female mice. Treatment with neem extract (Neem 50, Neem 100, Neem 250) and metformin (Met 250) significantly (^{#} p<0.05, ^{##} p<0.01, ^{###} p<0.001) attenuated the STZ-induced increased sugar level.

Table. Effect of Neem Extract on HbA1C Level of Streptozotocin-Induced Male and Female Diabetic Mice.

TABLE 3

| | | | | | |
|---|---|---|---|---|---|
| Vehicle Control | STZ | STZ + Neem 50 | STZ + Neem 100 | STZ + Neem 250 | STZ + Met 250 |
| (%) | (%) | (%) | (%) | (%) | (%) |

Male

| | | | | | | |
|---|---|---|---|---|---|---|
| Week 2 | 4.09 ± 0.45 | 5.99 ± 0.61* | 5.94 ± 0.28* | 6.28 ± 0.28* | 5.66 ± 0.14 | 5.05 ± 0.51 |
| Week 3 | 3.97 ± 0.11 | 6.03 ± 0.03* | 5.37 ± 0.56* | 5.48 ± 0.80 | 4.68 ± 0.97^{#} | 4.40 ± 0.65^{#} |

Female

| | | | | | | |
|---|---|---|---|---|---|---|
| Week 2 | 4.31 ± 0.35 | 6.02 ± 0.03* | 5.97 ± 0.79 | 5.16 ± 0.53 | 5.96 ± 0.09 | 5.53 ± 0.46 |
| Week 3 | 3.5 ± 0.29 | 5.97 ± 0.17** | 5.82 ± 0.11^{#} | 5.78 ± 0.06^{#} | 3.01 ± 0.44^{##} | 3.08 ± 0.58^{##} |

*p < 0.05,
**p < 0.01 in comparison to vehicle control;
^{#}p < 0.05,
^{##}p < 0.01 in comparison to STZ-induced of diabetic male and female mice

HbA1C (%) of male and female Neem+STZ and Metformin+STZ treated mice were compared with vehicle control mice and streptozotocin-induced diabetic mice. HbA1C level was significantly (*p<0.05). Treatment with neem extract (Neem 50, Neem 100, Neem 250) and metformin (Met 250) significantly (^{#} p<0.05, ^{##} p<0.01, ^{###} p<0.001) attenuated the STZ-induced increased in HbA1C level in STZ-induced diabetic male and female mice.

### Example 2 (Study 2)

Objective of Study 2: To study the effect of 3,8-dihydroxy-dibenzo-α-pyrone (Urolithin A) and 3-hydroxy-dibenzo-α-pyrone (Urolithin B), and their combination in particular ratios, on streptozotocin-induced diabetes in male and female mice.

Body weight, food intake, water intake, and fasting blood glucose (blood was collected following 12 hours fasting) were measured pre-induction of diabetes. Diabetes was induced by administering streptozotocin (50 mg/kg, body weight) in sodium citrate buffer, pH 4.5, intraperitoneally. Five days after streptozotocin administration fasting blood glucose was estimated (following 12 hours of fasting). Mice having blood glucose level 250±20 mg % was considered for the following study. The male and female mice were divided into the following four groups (n=8):

i. Control Vehicle (Con Veh) (0.3% CMC)

ii. Streptozotocin (STZ) Induced Diabetic Control

iii. 3-OH-DBP (Urolithin B)+STZ

iv. 3,8-(OH)₂-DBP (Urolithin A)+STZ

Following streptozotocin-induced hyperglycemia, treatment regimen was carried out for 1 month. 3-OH-DBP (Urolithin B) and 3,8-(OH)₂-DBP (Urolithin A) each was separately suspended in 0.3% CMC and administered at a dose of 10 mg/kg body weight orally for 1 month. During the 1-month treatment, body weight and food and water intake were monitored at regular intervals. FBG (following 12 hours of fasting) was estimated twice at 15-day intervals. The results were expressed in terms of mean±SEM (n=8). The data were subjected to one-way ANOVA followed by Tukey's test using GraphPad Prism 4.0 software to establish statistical significance (*p<0.05, **p<0.01, ***p<0.001; ^{#} p<0.05, ^{##} p<0.01, ^{###} p<0.001).

Table 5. Effect of 3-OH-DBP (Urolithin B) and 3,8-(OH)₂-DBP (Urolithin A) on Fasting Blood Glucose (Mg %) of Streptozotocin-Induced Diabetic Female Mice.

TABLE 5

| | | | 3-OH—DBP + | 3,8-(OH)₂—DBP + |
|---|---|---|---|---|
| | Vehical Control | STZ | STZ | STZ |
| No. of Days | (mg %) | (mg %) | (mg %) | (mg %) |
| Pre-Induction | 128.9 ± 3.466 | 123.5 ± 9.149 | 126.8 ± 3.848 | 95.45 ± 12.67 |
| Day 5 | 84.82 ± 11.30 | 276 ± 12.58*** | 251.6 ± 1.280*** | 253.2 ± 5.194*** |
| (Post-Induction) | | | | |
| Day 15 of | 108.2 ± 3.864 | 247 ± 6.840*** | 189.8 ± 2.690***^{, ###} | 166 ± 2.623***^{, ###} |
| treatment | | | | |
| Day 30 of | 119.7 ± 2.636 | 246.5 ± 6.018*** | 147.3 ± 15.21***^{, ###} | 153.9 ± 9.953***^{, ###} |
| treatment | | | | |

FBG of female 3-OH-DBP+STZ and 3,8-(OH)₂-DBP+STZ treated mice were compared with vehicle control mice and streptozotocin-induced diabetic mice. FBG was significantly (***p<0.001) increased in STZ group when compared with vehicle control group on day 5 (post-induction) indicating the hyperglycemic state of the animals. Treatment with 3-OH-DBP and 3,8-(OH)₂-DBP significantly (^{###} p<0.001) attenuated the STZ-induced increased sugar level, indicating antidiabetic activity.

Table 6. Effect of 3-OH-DBP and 3,8-(OH)₂-DBP on Fasting Blood Glucose of Streptozotocin-Induced Diabetic Male Mice.

The test results are listed in Table 6.

TABLE 6

| | Vehicle | | 3-OH—DBP + | |
|---|---|---|---|---|
| | Control | STZ | STZ | 3,8-(OH)₂—DBP + STZ |
| No. of Days | (mg %) | (mg %) | (mg %) | (mg %) |
| Pre- | 113.8 ± 5.031 | 98.64 ± 7.658*** | 99.06 ± 14.73*** | 94.12 ± 7.811*** |
| Induction | | | | |
| Day 5 | 108.9 ± 3.599 | 250.4 ± 13.31*** | 254.6 ± 1.575 | 252.2 ± 1.780 |
| (Post- | | | | |
| Induction) | | | | |
| Day 15 of | 110 ± 4.802 | 253.5 ± 8.879*** | 171 ± 6.586***^{, ###} | 166 ± 5.276***^{, ###} |
| treatment | | | | |
| Day 30 of | 123.7 ± 5.169 | 249.3 ± 4.122*** | 134.2 ± 3.381***^{, ###} | 156.7 ± 10.75***^{, ###} |
| treatment | | | | |

Fasting blood glucose of male 3-OH-DBP+STZ and 3,8-(OH)₂-DBP+STZ treated mice was compared with vehicle control mice and streptozotocin-induced diabetic mice.

FBG was significantly (***p<0.001) increased in STZ group when compared with vehicle control group on day 5 (post-induction) indicating the hyperglycemic state of the animals. Treatment with 3-OH-DBP and 3,8-(OH)₂-DBP, significantly (^{###} p<0.001) attenuated the STZ-induced increased sugar level, indicating antidiabetic activity.

### Example 3 (Study 3)

Objective of Study 3: To study the effect of neem extract, 3,8-dihydroxy-dibenzo-α-pyrone (Urolithin A), 3-hydroxy-dibenzo-α-pyrone (Urolithin B), and the combination of Urolithin A and Urolithin B in particular ratios, on blood glucose levels in genetically diabetic mice.

Five groups of diabetic mice (db/db, 11 weeks; n=3-5) were supplemented as follows:

A. Placebo

B. Neem extract (dose: 50 mg/kg)

C. 3-OH-DBP (Urolithin B) (dose: 2 mg/kg)

D. 3,8-(OH)₂-DBP (Urolithin A) (dose: 10 mg/kg)

E. A blend of 3,8-(OH)₂-DBP (Urolithin A) (dose: 10 mg/kg) and 3-OH-DBP (Urolithin B) (dose: 2 mg/kg)

Fasting blood glucose levels were monitored at regular intervals.

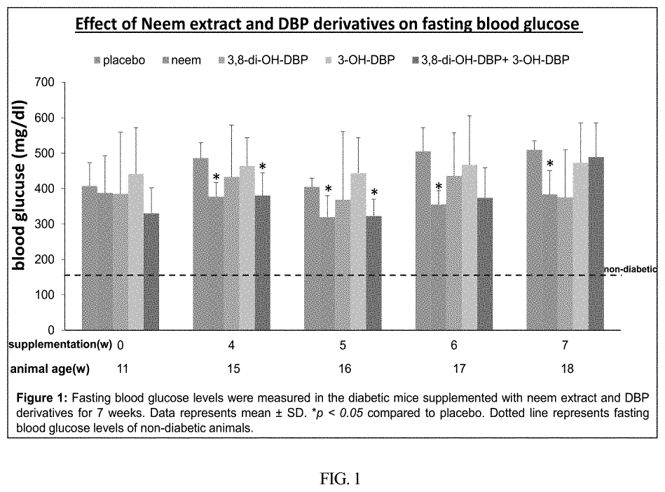

The results of the study, demonstrating the effect of neem extract and DBP derivatives (Urolithin A and Urolithin B) on fasting blood glucose in genetically diabetic mice, are depicted in FIG. 1.

Supplementation of neem extract (50 mg/kg) decreased the fasting blood glucose levels in db/db animals. Though the DBP derivatives alone did not show any changes in fasting blood sugar levels, a blend of 3,8-(OH)₂-DBP (dose: 10 mg/kg) and 3-OH-DBP (dose: 2 mg/kg) showed a significant decrease in the blood glucose level in initial stages. It should be noted that the mouse model used in this case (db/db; also known as Lepr^{db/db}) is a genetic mouse model that has a point mutation in the diabetes (db) gene encoding the leptin receptor gene. Hyperglycemia persists in these animals due to the genetic mutation, unlike in humans, in which diabetes develops mainly due to food habits and sedentary lifestyle. This might explain the reason for the bouncing back of the blend of 3,8-(OH)₂-DBP and 3-OH-DBP in the later stage.

In an embodiment, the combination of Urolithin A and Urolithin B can be prepared as a pharmaceutical or nutraceutical formulation. Exemplary wt./wt. ratios of Urolithin B to Urolithin A can range from about 0.2:1 to about 1:1. In a preferred embodiment, the wt./wt. ratios of Urolithin B to Urolithin A can range from about 0.2:1 to about 0.6:1.

In accordance with the examples as described herein, a daily dose of the aforementioned synergistic combination(s) of Urolithin B and Urolithin A can range from about 1.5 mg/kg to about 8.0 mg/kg in a human subject. In another embodiment, the daily dose can range from about 1.5 mg/kg to about 10.0 mg/kg in a human subject.

In one embodiment, a daily dose of the aforementioned synergistic combination(s) of Urolithin B and Urolithin A can range from about 100 mg to about 1000 mg in a human subject. In a preferred embodiment, a daily dose of the aforementioned synergistic combination(s) of Urolithin B and Urolithin A can range from about 100 mg to about 500 mg in a human subject.

It is further expected that the synergism observed in Study 3 will be exhibited in a similar manner in Studies 1 and 2, when carried out in a comparable dose range in a mammalian subject. It is further expected that the Urolithin B and Urolithin A synergy will be observed in a method for treating symptoms of diabetes in a human subject, including, but not limited to, increased blood glucose levels.

In an embodiment the neem extract can be prepared as a pharmaceutical or nutraceutical formulation.

In accordance with the examples as described herein, a daily dose of the neem extract can range from about 62.5 mg to about 3000 mg per day in a human subject. In another more preferred embodiment, the daily dose can range from about 125 mg to about 2000 mg per day in a human subject. In a most preferred embodiment, the daily dose can range from about 250 mg to about 1000 mg per day in a human subject.

It is further expected that the decrease in fasting blood glucose levels observed with neem extract in Study 3 will be observed in a method for treating symptoms of diabetes, including, but not limited to, increased blood glucose levels.

### Example 4—Clinical Study in Type 2 Diabetes

Evaluation of the effect of Neem extract on glycemic control, endothelial dysfunction, biomarkers and platelet aggregation in human subjects with type 2 diabetes mellitus.

Study Objectives

Primary: to study the effect of 12-week treatment with neem extract (NE) on fasting blood sugar (FBS), postprandial blood sugar (PPBS) and glycosylated hemoglobin (HbA1c) in patients with type 2 DM on metformin treatment.

Secondary: to study the effect on endothelial function and biomarkers including nitric oxide (NO), glutathione (GSH), Malondialdehyde (MDA), high sensitivity C-reactive protein (hsCRP), lipid profile, and platelet aggregation; to evaluate the effect on HOMA-IR and proinflammatory cytokines—IL6, TNF alpha; and to evaluate safety and tolerability.

Study Population

Type 2 DM patients on metformin therapy were recruited primarily from the General Medicine outpatient department of Nizam's Institute of Medical Sciences (NIMS). The study was conducted in the Dept. of Clinical Pharmacology and Therapeutics (CP&T, NIMS), Panjagutta, Hyderabad, India. Protocol no. CPT/04NEEM/DM/01.

Inclusion criteria as follows: male and female subjects between 30-65 years and willing to give informed consent and comply with protocol related study procedures; fasting plasma glucose of 110-126 mg/dL; glycosylated hemoglobin (HbA1c) between 6.5% and 8%; patients on a stable dose of anti-diabetic treatment (metformin 1500-2500 mg/day) for the past 8 weeks before the screening; patients with endothelial dysfunction—(salbutamol challenge test) documented as a decrease in RI index by ≤6%; and subjects not on any investigational products in the past 6 months.

Exclusion critera: subjects with abnormal hematological or biochemical parameters considered significant by the investigator; uncontrolled diabetes (HbA1c>8% and FBS>210 mg/dl); uncontrolled hypertension (SBP>180 mmHg and DBP>100 mmHg); serum Triglycerides>500 mg/dl; AST and ALT elevation>3 times upper limit of normal; serum creatinine more than 1.5 mg/dl; taking any other dietary or herbal supplements; or, any medical condition where the physician feels participation in the study could be detrimental to subjects well-being.

The study design is a randomized, double-blind, parallel-group study.

Study Treatment Groups

After screening, all the eligible patients were randomized to either of the four treatment groups in a double-blinded manner for 12 weeks, as follows.

Group A—One capsule of identical Placebo BID (each capsule containing microcrystalline cellulose 300 mg, croscarmellose sodium 20 mg, silicon dioxide-fumed 5 mg, magnesium stearate 5 mg), i.e., taken twice daily after food—morning and night.

Group B—One capsule containing 125 mg of neem extract (NE) BID (each capsule containing 125 mg of aqueous extract of neem, microcrystalline cellulose 175 mg, croscarmellose sodium 10 mg, silicon dioxide-fumed 3 mg, magnesium stearate 3 mg), i.e. taken twice daily after food—morning and night.

Group C—One capsule containing 250 mg of neem extract (NE) BID (each capsule containing 250 mg of aqueous extract of neem, microcrystalline cellulose 50 mg, croscarmellose sodium 10 mg, silicon dioxide-fumed 3 mg, magnesium stearate 3 mg), i.e. taken twice daily after food—morning and night.

Group D—One capsule containing 500 mg of neem extract BID (NE) (each capsule containing 500 mg of aqueous extract of neem, microcrystalline cellulose 50 mg, croscarmellose sodium 10 mg, silicon dioxide-fumed 3 mg, magnesium stearate 3 mg), i.e. taken twice daily after food—morning and night.

All treatments were used as add-on therapy to the existing treatment with metformin.

The placebo capsules and the neem extract capsules were supplied by Natreon, Inc., New Brunswick, N.J., USA.in

Neem extract as used herein is a standardized aqueous extract of Azadirachta indica (leaf) available as PhytoBGS® from Natreon, Inc., comprising about 2.3-3.7% w/w (av. 3.0% w/w) total flavonoids (incl. quercetin-3-O-glucoside, quercetin-3-O-rutinoside, apegenin rutinoside, rutin derivatives), about 7.3-11.3% w/w (av. 9.3% w/w) myo-inositol monophosphate, about 5.2-7.7% w/w (av. 6.6% w/w) total polyphenols, and 6.3-13.1% w/w (av. 10.1% w/w) total amino acids (incl. Arginine and Methionine). Specification: not less than about 7% by wt. myo-inositol monophosphate and not less than about 1% by wt. flavonoids.

Outcome Measures

Primary: Reduction in HbA1c by at least 1% with 12 weeks of therapy; and reduction in fasting blood glucose (FBG) and post-prandial blood glucose (PPBG) by at least 10 mg/dl.

Secondary: Change in endothelial dysfunction as assessed by more than 6% change in reflection index at 12 weeks in all the treatment groups; Change in oxidative stress biomarkers nitric oxide (NO), glutathione (GSH), malondialdehyde (MDA), inflammatory biomarker—high sensitivity C-reactive protein (hsCRP), and platelet aggregation from baseline to end of 12 weeks therapy in all the treatment groups; Change in insulin resistance (HOMA-IR) and proinflammatory cytokines—IL6, TNF alpha with 12 weeks treatment; and safety and tolerability assessment of the test medications.

Methodology

Subjects were screened after signing the written informed consent. Eighty eligible subjects were randomized in equal ratios to the four test formulations respectively: group A (Placebo BD=BID), group B (NE 125 mg BD), group C (NE 250 mg BD) and group D (NE 500 mg BD). All subjects enrolled were on a stable dose of concomitant medication over the past 8 weeks as prescribed by the physician, and the same concomitant medication was continued throughout the study.

Subjects were on treatment with either one of the four study medications for 12 weeks from the randomization visit. The screening was done at Visit 1. Randomization (visit 2) was done within one week of visit 1. Visit 3 and 4 were follow-up visits after four and eight weeks of therapy. Visit 5 (End of treatment) was at the end of 12 weeks of treatment. Patients reported to the department under fasting condition at every visit. At screening or visit 1, after describing in detail about the study, written informed consent was taken from each subject for participation.

Then the subject's demography data, medical history, and vitals were recorded. Evaluation of endothelial function was done non-invasively by Micro Medical system using salbutamol challenge test. Subjects reported to the research unit under fasting condition. Blood samples were drawn for fasting blood sugar, HbA1c, safety assessments, lipid profile, hs-CRP, biomarker estimations, platelet aggregation, HOMA-IR and proinflammatory cytokines—IL6, TNF alpha. The subjects were provided with breakfast. Two hours after breakfast, a blood sample was taken for estimation of postprandial blood sugar. The subjects were asked to report to the unit within the next week for visit 2.

Visit 2 was the randomization visit. Subjects fulfilling the inclusion/exclusion criteria were randomized to receive one of the four treatment as per prior randomization schedule. Vitals were recorded, and physical examination performed. Subjects were enquired of regarding the use of any concomitant medication and adverse events, and the same was recorded in case record form. The subjects were dispensed study medication as per the randomization schedule and asked to come for the next follow up visit after four weeks.

At the subsequent follow-up visits, visit 3 (week 4) and visit 4 (week 8) subjects were reviewed. Vitals were recorded. Physical examination was performed. Any alteration in concomitant medication, any adverse drug reactions were reported and noted in CRF. Compliance to study medication was assessed by pill count at each visit. Endothelial dysfunction was evaluated by salbutamol challenge test and blood samples collected for estimation of fasting and postprandial (PP) blood sugars (2 hours after breakfast). Any ADR, especially signs, and symptoms of hypoglycemia were enquired and recorded in case report form (CRF). Study medication was dispensed as per the schedule.

At visit 5 (week 12), which was the end of the treatment period, vitals were recorded, and physical examination was performed. Compliance to study medication was assessed by pill count. Any adverse drug reactions reported were recorded. Endothelial function (RI) was assessed, and the blood sample was collected for the estimation of all the biomarkers and safety assessment.

FBG, PPBG, biomarkers (MDA, NO, GSH, hsCRP), reflection index (RI) were estimated at baseline, and each visit. HOMA-IR, IL-6, and TNF alpha were evaluated at baseline, 4, and 12 weeks. HbA1c, platelet aggregation, and lipid profile were estimated at baseline and 12 weeks of treatment.

Methods of Assessment

Assessment of Endothelial function: Salbutamol Challenge Test. A salbutamol challenge test employing digital volume plethysmography was used to assess endothelial function as reported by Chowienczyk et al., “Photoplethysmographic assessment of pulse wave reflection: blunted response to endothelium dependant beta 2-adrenergic vasodilation in type 2 diabetes mellitus,” J. Am. Coll. Cardiol. (1999 December) 34(7):2007-14; and Naidu, et al., “Comparison of two β2 adrenoceptor agonists by different routes of administration to assess human endothelial function,” Indian J. Pharmacol. (2007) 39:168-9. Patients were examined in the supine position after 5 minutes of rest. A digital volume pulse (DVP) was obtained using photoplethysmograph (Pulse Trace PCA2, PT200, Micro Medical, Kent, UK) transmitting infrared light at 940 nm, placed on the index finger of the right hand. The signal from the plethysmograph was digitized using a 12-bit analog to digital converter with a sampling frequency of 100 Hz. DVP waveforms were recorded over 20 second period, and the height of the late systolic/early diastolic portion of the DVP was expressed as a percentage of the amplitude of the DVP to yield the reflection index (RI), as per the procedure described in detail by Millasseau et al., “Determination of age related increases in large artery stiffness by digital pulse contour analysis,” Clinical Science (2002) 103: 371-377. DVP recordings were taken, three measurements of reflection index (RI) was calculated, and the mean value determined. Patients were administered 400 mcg of salbutamol by inhalation. After 15 minutes, three measurements of RI were obtained again, and the difference in mean RI before and after administration of salbutamol was used for assessing endothelial function. A change of <6% in RI post salbutamol was considered as endothelial dysfunction. Results are presented in the study data summary Table 8 below.

Evaluation Of Biomarkers; Platelet Aggregation Test; Special Tests And Safety Parameters

Nitric oxide levels were estimated spectrophotometrically as described in Miranda, et al., “A Rapid, Simple Spectrophotometric Method for Simultaneous Detection of Nitrate and Nitrite,” NITRIC OXIDE: Biology and Chemistry (2001) Vol. 5, No. 1, pp. 62-71. The levels of MDA and Glutathione were estimated spectrophotometrically as described in Vidyasagar, et al., “Oxidative stress and antioxidant status in acute organophosphorous insecticide poisoning,” Indian J. Pharmacol. (April 2004) 36(2): 76-79, and G. L. Ellman, Arch. Biochem. Biophys. (1959) 82: 70-77 (original determination), respectively. hsCRP (high sensitivity C-reactive protein) was determined by ELISA method. Samples were collected after an overnight fast for determination of hemoglobin, blood urea, and serum creatinine, liver function test, lipid profile [total cholesterol, high-density lipoprotein cholesterol (HDL-C), low-density lipoprotein cholesterol (LDL-C), very low-density lipoprotein (VLDL-C) and Triglycerides] using appropriate standard techniques. Platelet aggregation test with ADP and Collagen were done using platelet aggregometry test, i.e., (Chronolog Light Transmittance Aggregometry). TNF alpha and IL6 were estimated using commercially available ELISA kits. The HOMA-IR (Homeostatic Model Assessment for Insulin Resistance) is an approximating equation for insulin resistance. It is estimated using the formula Fasting insulin (mIU/L)×Fasting glucose (mg/dL)/405. HOMA-IR value less than 3 indicates normal insulin resistance; values between 3-5 indicate moderate insulin resistance, whereas values above 5 indicate severe insulin resistance. See results in the study data summary Table 8 below.

Data analysis. Data is expressed as mean+SD. The within-group analysis was done using paired ‘t’ test. Between the groups, analyses was done using ANOVA. Post-hoc analysis between the groups was done with Tukey's test. A p-value<0.05 was considered statistically significant. The statistical analysis was done using the software GraphPad Prism 8.

Sample size: To detect a reduction of 10 mg/dL of PPBG with a 5% margin of alpha error, power of 80% and assuming a dropout rate of 10% and a screen failure of 5% a total of 94 patients were screened.

Results of Study

A total of 94 subjects were screened, and 80 eligible subjects enrolled in the study. A total of 78 subjects completed 12 weeks of treatment. Two subjects from group C (NE 250 mg BD) dropped out of the study before the first follow-up. One subject dropped out as he got transferred to another city. The other subject dropped out citing logistical reasons as he had relocated to a far-off location in the same city. The demographic data in Table 1 shows all the subjects who were randomized, hence 20 in each group. Further, Table 2 to Table 14 includes the data of the subj ects who have completed the study. Thus, a total of 20 subjects in group A (Placebo BD), 20 subjects in group B (NE 125 mg BD), 18 subjects in group C (NE 250 mg BD) and 20 subjects in group D (NE 500 mg BD) completed 12 weeks of study treatment.

The demographic characteristics of the four study groups are depicted in Table 7.

There were no significant differences between treatment groups in baseline characteristics, including age and body mass index (BMI) indicating a homogenous population.

TABLE 7
Demographic data

| | | NE 125 | NE 250 | NE 500 |
|---|---|---|---|---|
| | Placebo BD | mg BD | mg BD | mg BD |
| | (A) | (B) | (C) | (D) |
| Total No. | 20 | 20 | 20 | 20 |
| Gender (M/F) | 13M/7F | 8M/12F | 14M/6F | 11M/9F |
| Age (Years) | 51.80 ± 6.69 | 54.50 ± 8.18 | 53.20 ± 7.67 | 55.05 ± 7.88 |
| BMI (Kg/m2) | 26.54 ± 2.33 | 25.63 ± 2.92 | 24.51 ± 1.91 | 26.01 ± 1.84 |

TABLE 8
Summary of the results from Type 2 Diabetes study at 12 weeks compared to placebo

| | % Change in 12 Weeks & p-Value Compared to Placebo | | | |
|---|---|---|---|---|
| | Placebo | 125 mg BID | 250 mg BED | 500 mg BED |
| Parameter | 13M + 7F | 8M + 12F | 14M + 6F | 11M + 9F |
| Fasting blood glucose level | −1.3 ± 3.4 | −8.3 ± 4.1 | −10.1 ± 3.7 | −19.3 ± 3.9 |
| (FBG) | NS | p ≤ 0.0001 | p ≤ 0.0001 | p ≤ 0.0001 |
| Post-prandial blood glucose | −0.6 ± 2.3 | −10.8 ± 4.8 | −15.6 ± 4.7 | −22.6 ± 4.4 |
| level (PPBG) | NS | p ≤ 0.0001 | p ≤ 0.0001 | p ≤ 0.0001 |
| Insulin resistance | −10.1 ± 7.9 | −23.0 ± 1.1 | −32.9 ± 12.26 | −57.4 ± 6.4 |
| (HOMA-IR) | p ≤ 0.01 | p ≤ 0.001 | p ≤ 0.0001 | p ≤ 0.0001 |
| Glycosylated hemoglobin | −0.7 ± 1.3 | −3.4 ± 1.6 | −8.7 ± 3.2 | −19.6 ± 4.9 |
| (HbA1c) | p ≤ 0.05 | p ≤ 0.0001 | p ≤ 0.0001 | p ≤ 0.0001 |
| Reflection Index | −0.10 ± 1.00 | −0.84 ± 0.31 | −2.41 ± 1.93 | −3.21 ± 0.89 |
| (RI, %) | NS | NS | p ≤ 0.0001 | p ≤ 0.0001 |
| Glutathione | 0.10 ± 0.39 | 1.17 ± 0.34 | 8.14 ± 1.49 | 12.14 ± 2.39 |
| (GSH) | NS | NS | p ≤ 0.0001 | p ≤ 0.0001 |
| Malondialdehyde | −0.25 ± 0.57 | −2.90 ± 2.16 | −3.18 ± 1.33 | −11.47 ± 3.50 |
| (MDA) | NS | NS | NS | p ≤ 0.05 |
| Nitric oxide | 5.39 ± 12.58 | 13.40 ± 5.05 | 16.28 ± 7.95 | 22.12 ± 7.25 |
| (NO) | NS | NS | NS | NS |
| C-Reactive protein | −0.59 ± 1.31 | −11.39 ± 7.72 | −18.28 ± 6.93 | −23.86 ± 12.10 |
| (hsCRP) | NS | p ≤ 0.05 | p ≤ 0.05 | p ≤ 0.0001 |
| Interleukin-6 | −0.08 ± 0.24 | −3.86 ± 5.03 | −8.10 ± 4.87 | −10.31 ± 8.87 |
| (IL-6) | NS | p ≤ 0.0001 | p ≤ 0.0001 | p ≤ 0.0001 |
| Tumor necrosis factor | −0.06 ± 1.42 | −5.11 ± 3.37 | −7.27 ± 4.40 | −9.09 ± 6.72 |
| (TNF-α) | NS | NS | NS | NS |

P values are for the test product vs placebo

### CONCLUSIONS

In the present study, as shown in Table 8, the aqueous extract of neem significantly decreased the levels of FBG, PPBG, HbA1c, and HOMA-IR. It also significantly improved the biomarkers of stress MDA, NO, and GSH along with endothelial function as estimated by improvement in reflection index (RI). Marked improvement in the levels of hsCRP, IL6, and TNF alpha was also seen. Measurements were made at 4, 8 and 12 weeks.

Significant improvement in the levels of FBG, PPBG, HbA1c, and HOMA-IR was observed with group B (NE 125 mg BD), group C (NE 250 mg BD) and group D (NE 500 mg BD) at the end of 4, 8 and 12 weeks when compared to baseline and placebo. No effect was seen with group A (Placebo BD). Between-group analysis, it was observed though group C and group D were better than group B, group D demonstrated better response than group C in the above parameters.

Endothelial dysfunction measured by the estimation of RI showed significant improvement with group B, group C, and group D at 4, 8, and 12 weeks when compared to baseline. Group A did not show any effect. On further analysis between groups, it was observed that group C and group D showed better response than group B with group D demonstrating maximum improvement in RI.

Biomarkers of oxidative stress MDA, NO and GSH also improved significantly.

The GSH values with group B, group C, and group D improved significantly at 4, 8, and 12 weeks of treatment when compared to baseline. Group A did not show any effect. Between-group analysis at 4 and 8 weeks showed the significance for group B, group C, and group D when compared to placebo. At 12 weeks, significance was noted for group C and group D when compared to Placebo and treatment groups.

The levels of MDA have shown significant improvement with group B, group C, and group D at 4, 8, and 12 weeks of treatment when compared to baseline. No statistically significant improvement was seen in group A. Between-group analysis at 4 and 8 weeks did not show any significance in the improvement of MDA levels with all the treatment groups when compared to placebo. At week 12, only group D has shown a significant effect when compared to placebo. No significance was observed between the treatment groups.

Nitric oxide levels improved significantly with group B, group C, and group D at 4, 8, and 12 weeks of treatment when compared to baseline. Group A did not show any significance. No statistical significance was seen when a between-group comparison was done for NO with placebo and treatment groups.

Improvement in the levels of inflammatory marker hsCRP was observed at 4, 8, and 12 weeks with group B, group C, and group D when compared to baseline. Group A had not shown any improvement. Between-group analysis of hsCRP showed that at 4 and 8 weeks group B and group C did not show any significance when compared to placebo. Whereas group D had shown significance when compared to placebo and other treatment groups. At week 12, group B, group C, and group D have shown significant improvement in hsCRP levels when compared to placebo. Group D had shown maximum response when compared to placebo and other treatment groups.

Improvement in the levels of pro-inflammatory marker IL-6 was observed at 4 and 12 weeks with group B, group C, and group D when compared to baseline. Group A had not shown any improvement. The between-group analysis was significant for group B, group C, and group D at both 4 and 12 weeks when compared to placebo. No significance was observed between the treatment groups.

Pro-inflammatory marker TNF alpha levels also showed significant improvement at 4 and 12 weeks with group B, group C, and group D when compared to baseline. No effect was seen in the placebo group. Between-group analysis of TNF alpha showed no significance with placebo and among the treatment groups.

Platelet aggregation percent inhibition (% inhibition) at the end of 12 weeks of treatment was determined. The normal range of platelet aggregation using ADP (10 iIM/ml) and collagen (2 μg/ml) is between 60% and 90%. To show if the drug affects platelet aggregation, there should be more than 30% change in percent inhibition. No effect on platelet aggregation was observed with any of the treatment groups.

All safety haematological and biochemical parameters performed as per visit schedule were within normal limits with all the four treatment groups at baseline and the end of the treatment. One patient in group B (NE 125 mg BD) and one in group D (NE 500 mg BD) reported mild GI disturbance, which subsided with symptomatic treatment. None of the subject in either group discontinued the study due to adverse events.

The medications used in the study were well tolerated. No serious adverse events were reported. None of the subjects discontinued the study due to any adverse events, which suggests the favorable safety profile of the treatments used in the study.

The findings of the present study indicate that the aqueous extract of neem may be useful in the management of hyperglycemia in type 2 DM patients as an add-on medication. Further, it appears to improve the cardiovascular complications of type 2 DM, as evidenced by changes in RI and biomarkers.

Table 9 depicts a summary of the PhytoBGS T2DM study (NIMS) data indicating Baseline (B) & Mean % Change (M % C) in 12 weeks.

TABLE 9
Baseline (B) & Mean % Change (M % C) in 12 weeks

| | FBG | | PPBG | | HOMA IR | | HbA1c | |
|---|---|---|---|---|---|---|---|---|
| Groups | B | M % C | B | M % C | B | M % C | B | M % C |
| Placebo | 121.8 | −1.3 | 202.3 | −0.6 | 4.9 | −10.1 | 7.62 | −0.7 |
| BD | | | | | | | | |
| (A) | | | | | | | | |
| 125 mg | 119.2 | −8.3 | 194.4 | −10.8 | 4.5 | −23.0 | 6.87 | −3.4 |
| BD | | | | | | | | |
| (B) | | | | | | | | |
| 250 mg | 115.5 | −10.1 | 192.3 | −15.6 | 3.8 | −32.9 | 7.52 | −8.7 |
| BD | | | | | | | | |
| (C) | | | | | | | | |
| 500 mg | 120.7 | −19.3 | 205.9 | −22.6 | 4.6 | −57.4 | 7.78 | −19.6 |
| BD | | | | | | | | |
| (D) | | | | | | | | |

p Values are for the test product vs placebo and significant at p ≤ 0.0001, except for HOMA IR value for 125 mg BID group (p ≤ 0.001).

It should be noted that administration of neem extract over 12 weeks decreased FBG, PPBG and HbA1C levels in type 2 diabetes patients, on top of metformin, bringing these levels to near normal. Measured values post treatment were as follows: FBG: 97.3±3.7 (p<0.0001); PPBG: 159.3±7.1 (p<0.0001); HbA1C: 6.26±0.4 (p<0.0001).

### Example 5—Clinical Study in Metabolic Syndrome

Evaluation of the effect of Neem extract on glycemic control, endothelial dysfunction, biomarkers and platelet aggregation in human subjects with metabolic syndrome.

The study methodology and treatment levels and treatment groups were the same as in Example 4, except that in this study subjects meeting metabolic syndrome guidelines were enrolled.

The demographic characteristics of the four study groups are depicted in Table 10.

There were no significant differences between treatment groups in baseline characteristics, including age and body mass index (BMI) indicating a homogenous population.

TABLE 10
Demographic data

| | | NE 125 | NE 250 | NE 500 |
|---|---|---|---|---|
| | Placebo BD | mg BD | mg BD | mg BD |
| | (A) | (B) | (C) | (D) |
| Total No. | 20 | 20 | 20 | 20 |
| Gender (M/F) | 11M/9F | 13M/7F | 12M/8F | 11M/9F |
| Age (Years) | 42.7 ± 5.42 | 43.45 ± 5.79 | 47.35 ± 5.14 | 46.8 ± 5.16 |
| BMI (Kg/m2) | 30.74 ± 0.76 | 31.21 ± 1.09 | 31.01 ± | 31.10 ± 1.07 |
| | | | 1.0497.3 ± 3.7 | |

TABLE 11
Summary of the results from the
metabolic syndrome study compared to placebo

| | Mean % Change in 12 Weeks & p-Value | | | |
|---|---|---|---|---|
| | Compared to Placebo | | | |
| | Placebo | 125 mg BID | 250 mg BID | 500 mg BID |
| Parameter | 11M + 9F | 13M + 7F | 12M + 8F | 11M + 9F |
| Fasting blood | 5.7 ± 5.5 | 2.4 ± 8.3 | −3.1 ± 3.9 | −6.0 ± 2.5 |
| glucose level | | | | Ω-p ≤ 0.01 |
| (FBG) | | | | |
| Post-prandial | 4.4 ± 2.6 | 3.6 ± 4.2 | −0.4 ± 5.7 | −4.1 ± 4.6 |
| blood glucose | | | | Ω-p ≤ 0.01 |
| level (PPBG) | | | | |
| Reflection Index | −0.07 ± | −0.59 ± | −0.68 ± | −1.15 ± |
| (Endothelial | 0.15 | 0.25 | 0.29 | 0.28 |
| Function) | | ‡-p ≤ 0.05 | ‡-p ≤ 0.05 | Ω-p ≤ 0.01 |

Table 11 shows blood glucose and reflection index data summary compared to placebo (between the groups) after 12 weeks of treatment.

TABLE 12
Summary of the results from the metabolic syndrome study
compared to baseline

| | Mean % Change in 12 Weeks & p-Value | | | |
|---|---|---|---|---|
| | Compared to Baseline | | | |
| | Placebo | 125 mg BID | 250 mg BID | 500 mg BID |
| Parameter | 13M + 7F | 8M + 12F | 14M + 6F | 11M + 9F |
| HOMA IR | 24.9 ± 18.4 | 4.3 ± 15.2 | −5.6 ± 9.5 | −12.1 ± 7.1 |
| | | | $-p ≤ 0.01 | *-p ≤ 0.0001 |
| HbA1c | 1.28 ± 1.25 | 0.47 ± 1.47 | −0.58 ± 0.98 | −1.72 ± 2.30 |
| | | | $-p ≤ 0.01 | $-p ≤ 0.01 |
| Glutathione | 0.06 ± 0.14 | 0.41 ± 0.15 | 0.56 ± 0.11 | 0.76 ± 0.45 |
| (GSH) | | *-p ≤ 0.0001 | *-p ≤ 0.0001 | *-p ≤ 0.0001 |
| Malondialdehyde | 3.65 ± 2.04 | −1.03 ± 2.44 | −3.05 ± 2.31 | −5.88 ± 3.52 |
| (MDA) | | | *-p ≤ 0.0001 | *-p ≤ 0.0001 |
| Nitric oxide | 1.35 ± 4.46 | 1.39 ± 1.42 | 3.42 ± 9.32 | 4.14 ± 2.31 |
| (NO) | | | *-p ≤ 0.0001 | *-p ≤ 0.0001 |
| C-Reactive | 3.78 ± 5.99 | 2.69 ± 4.13 | −3.30 ± 1.82 | −10.22 ± 7.85 |
| protein | | | *-p ≤ 0.0001 | *-p ≤ 0.0001 |
| (hsCRP) | | | | |
| Interleukin-6 | 0.43 ± 1.54 | −0.62 ± 2.58 | −2.70 ± 4.28 | −5.18 ± 3.40 |
| (IL-6) | | | $-p ≤ 0.01 | *-p ≤ 0.0001 |
| Tumor necrosis | 0.58 ± 0.94 | −0.75 ± 2.59 | −0.99 ± 0.73 | −1.01 ± 0.58 |
| factor-α | | | *-p ≤ 0.0001 | *-p ≤ 0.0001 |
| (TNF-α) | | | | |
| Platelet | No effect | No effect | No effect | No effect |
| Aggregation | | | | |
| Inhibition | | | | |

Table 12 shows oxidative stress biomarkers and other blood measurement data summary compared to baseline (within the group) after 12 weeks of treatment.

The nutraceutical compositions of the present invention may be administered in combination with a nutraceutically acceptable carrier. The active ingredients in such formulations may comprise from 1% by weight to 99% by weight, or, alternatively, 0.1% by weight to 99.9% by weight. “Nutraceutically acceptable carrier” means any carrier, diluent, or excipient that is compatible with the other ingredients of the formulation and not deleterious to the user. In accordance with one embodiment, suitable nutraceutically acceptable carriers can include ethanol, aqueous ethanol mixtures, water, fruit, and/or vegetable juices, and combinations thereof.

The pharmaceutical compositions of the present invention may be administered in combination with a pharmaceutically acceptable carrier. The active ingredients in such formulations may comprise from 1% by weight to 99% by weight, or, alternatively, 0.1% by weight to 99.9% by weight. “Pharmaceutically acceptable carrier” means any carrier, diluent, or excipient that is compatible with the other ingredients of the formulation and not deleterious to the user.

Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films, or oral dissolvable strips can be used. Other useful delivery systems comprise oral or nasal sprays or inhalers, and the like.

For oral administration, Shilajit, Urolithin A, Urolithin B, or combinations thereof, may be further combined with one or more solid inactive ingredients for the preparation of tablets, capsules, pills, powders, granules, or other suitable dosage forms. For oral administration, neem extract may be further combined with one or more solid inactive ingredients for the preparation of tablets, capsules, pills, powders, granules, or other suitable dosage forms. For example, the active agent may be combined with at least one excipient such as fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like.

The components of the invention, together with a conventional adjuvant, carrier, or diluent, may thus be placed into the form of pharmaceutical compositions and unit dosages thereof. Such forms include: solids, in particular, tablets, filled capsules, powder, and pellet forms; and liquids, in particular, aqueous or non-aqueous solutions, suspensions, emulsions, elixirs, and capsules filled with the same, all for oral use; suppositories for rectal administration; and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The components of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired.

The powders and tablets preferably contain from five or ten to about seventy percent of the active compound(s). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is thus in association with it. Similarly, cachets and lozenges are included. Tablets, powders, capsules, pills, cachets, and lozenges are included. Tablets, powders, capsules, pills, cachets, and lozenges can be used as solid forms suitable for oral administration.

Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g., by injection, for example, bolus injection or continuous infusion) and may be presented in unit dose form in ampoules, pre-filled syringes, small-volume infusion, or in multi-dose containers with an added preservative. The compositions may take such forms as suspensions, solutions, or emulsions in oily or aqueous vehicles, and may contain formulation agents, such as suspending, stabilizing, and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Compositions suitable for topical administration in the mouth includes lozenges comprising the active agent in a flavored base, usually sucrose and acacia or tragacanth; pastilles comprising the active ingredient in an inert base such as gelatin and glycerine or glucose and acacia; and mouthwashes comprising the active ingredient in suitable liquid carrier.

Solutions or suspensions are applied directly to the nasal cavity by conventional means, for example, with a dropper, pipette, or spray. The compositions may be provided in single or multi-dose form. In compositions intended for administration to the respiratory tract, including intranasal compositions, the compound will generally have a small particle size, for example, of the order of 5 microns or less. Such a particle size may be obtained by means known in the art, for example, by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets, capsules, and lozenges for oral administration and liquids for oral use are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of Remington's Pharmaceutical Sciences (Mack Publishing Co., Easton, Pa.).

Solid nutritional compositions for oral administration may optionally contain, in addition to the above enumerated nutritional composition, ingredients, or compounds: carrier materials, such as corn starch, gelatin, acacia, microcrystalline cellulose, kaolin, dicalcium phosphate, calcium carbonate, sodium chloride, alginic acid, and the like; disintegrators, including microcrystalline cellulose, alginic acid, and the like; binders, including acacia, methylcellulose, sodium carboxymethylcellulose, polyvinylpyrrolidone, hydroxypropyl methylcellulose, ethyl cellulose, and the like; and lubricants, such as magnesium stearate, stearic acid, silicone fluid, talc, waxes, oils, colloidal silica, and the like. The usefulness of such excipients is well known in the art.

In one preferred embodiment, the nutritional composition may be in the form of a liquid. In accordance with this embodiment, a method of making a liquid composition is provided.

Liquid nutritional compositions for oral administration in connection with a method for preventing and/or treating inflammation, colds, and/or flu can be prepared in water or other aqueous vehicles. In addition to the above-enumerated ingredients or compounds, liquid nutritional compositions can include suspending agents such as, for example, methylcellulose, alginates, tragacanth, pectin, kelgin, carrageenan, acacia, polyvinylpyrrolidone, polyvinyl alcohol, and the like. The liquid nutritional compositions can be in the form of a solution, emulsion, syrup, gel, or elixir, including or containing, together with the above enumerated ingredients or compounds, wetting agents, sweeteners, and coloring and flavoring agents. Various liquid and powder nutritional compositions can be prepared by conventional methods. Various ready-to-drink formulations (“RTDs”) are contemplated.

Routes of Administration

The compositions may be administered by any suitable route, including, but not limited to, oral, sublingual, buccal, ocular, pulmonary, rectal, and parenteral administration, or as an oral or nasal spray (e.g., inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the installation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection, or infusion) administration, or those in a form suitable for administration by inhalation or insufflations, including powders and liquid aerosol administration, or by sustained release systems. Suitable examples of sustained release systems include semipermeable matrices of solid hydrophobic polymers containing the compound of the invention, which matrices may be in the form of shaped articles, e.g., films or microcapsules.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately +10%; in other embodiments, the values may range in value either above or below the stated value in a range of approximately +5%; in other embodiments, the values may range in value either above or below the stated value in a range of approximately +2%; in other embodiments the values may range in value either above or below the stated value in a range of approximately +1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entireties. The present invention may be embodied in other specific forms without departing from the spirit or essential attributes thereof, and, accordingly, reference should be made to the appended claims, rather than to the foregoing specification, as indicating the scope of the invention.

We claim:

1. A method for treating or preventing type 2 diabetes mellitus comprising administering to an individual in need thereof a therapeutically effective amount of a composition comprising an aqueous extract of Azadirachta indica, wherein post-prandial blood glucose or fasting blood glucose is reduced.

2. The method of claim 1, wherein the blood glucose is reduced by about 20% compared to placebo.

3. The method of claim 1, wherein the aqueous extract of Azadirachta indica is provided in a daily dose range of from about 62.5 mg to about 3000 mg.

4. The method of claim 1, wherein the aqueous extract of Azadirachta indica is provided in a daily dose range of from about 125 mg to about 2000 mg.

5. The method of claim 1, wherein the aqueous extract of Azadirachta indica is provided in a daily dose range of from about 250 mg to about 1000 mg.

6. The method of claim 1, wherein the wherein the aqueous extract of Azadirachta indica is administered over about 8 weeks to about 12 weeks.

7. The method of claim 1, wherein the aqueous extract includes about 3% by weight total flavonoids and not less than 7% by weight myo-inositol monophosphate, based on the total weight of the composition.

8. The method of claim 1, further wherein HbA1c is reduced by about 20% compared to placebo.

9. The method of claim 1, further wherein insulin resistance as measured by HOMA-IR is reduced by greater than 50% compared to placebo.

10. A method of treating or preventing endothelial dysfunction in a diabetic individual suffering from type 2 diabetes mellitus comprising administering to the individual in need thereof a therapeutically effective amount of a composition comprising an aqueous extract of Azadirachta indica, wherein endothelial function is improved.

11. The method of claim 10, wherein the improved endothelial function includes an increase of at least about 10-12% in the blood level of nitric oxide (NO) in the diabetic individual.

12. The method of claim 10, wherein the improved endothelial function includes an increase of at least about 15-20% in the blood level of glutathione (GSH) in the diabetic individual.

13. The method of claim 10, wherein the improved endothelial function includes a decrease of at least about 8-12% in the blood level of malondialdehyde (MDA) in the diabetic individual.

14. The method of claim 10, wherein the improved endothelial function includes a decrease of at least about 20% in the blood level of C-Reactive protein (hsCRP) in the diabetic individual.

15. The method of claim 10, wherein the improved endothelial function includes a decrease of at least about 10% in the blood level of Interleukin-6 (IL-6) in the diabetic individual.

16. The method of claim 10, wherein the improved endothelial function includes a decrease of at least about 9% in the blood level of TNF-alpha in the diabetic individual.

17. The method of claim 10, wherein the aqueous extract of Azadirachta indica is provided in a daily dose range of from about 62.5 mg to about 3000 mg.

18. The method of claim 10, wherein the aqueous extract of Azadirachta indica is provided in a daily dose range of from about 125 mg to about 2000 mg.

19. The method of claim 10, wherein the aqueous extract of Azadirachta indica is provided in a daily dose range of from about 250 mg to about 1000 mg.

20. The method of claim 10, wherein the wherein the aqueous extract of Azadirachta indica is administered over about 8 weeks to about 12 weeks.

21. The method of claim 10, wherein the aqueous extract includes about 3% by weight total flavonoids and not less than 7% by weight myo-inositol monophosphate, based on the total weight of the composition.

22. A method for treating or preventing symptoms of metabolic syndrome comprising administering to an individual in need thereof a therapeutically effective amount of a composition comprising an aqueous extract of Azadirachta indica, wherein post-prandial blood glucose or fasting blood glucose is reduced.

23. The method of claim 22, wherein the blood glucose is reduced by about 4-6% compared to placebo.

24. The method of claim 22, wherein the aqueous extract of Azadirachta indica is provided in a daily dose range of from about 62.5 mg to about 3000 mg.

25. The method of claim 22, wherein the aqueous extract of Azadirachta indica is provided in a daily dose range of from about 125 mg to about 2000 mg.

26. The method of claim 22, wherein the aqueous extract of Azadirachta indica is provided in a daily dose range of from about 250 mg to about 1000 mg.

27. The method of claim 22, wherein the aqueous extract of Azadirachta indica is administered over about 8 weeks to about 12 weeks.

28. The method of claim 22, wherein the aqueous extract includes about 3% by weight total flavonoids and not less than 7% by weight myo-inositol monophosphate, based on the total weight of the composition.

29. The method of claim 22, further wherein HbA1c is reduced by about 2% in the individual compared to baseline.

30. The method of claim 22, further wherein insulin resistance as measured by HOMA-IR is reduced by greater than 10% in the individual compared to baseline.

31. The method of claim 22, further wherein endothelial function is improved including a decrease of at least about 10% in the blood level of C-Reactive protein (hsCRP) in the individual.
